(19) United States
(12) Patent Application Publication
(10) Pub. No.: US 2021/0138452 A1
(43) Pub. Date: May 13, 2021

(54) DEVICE AND METHOD FOR DETECTING A SPECIFIC ANALYTE IN A LIQUID SAMPLE AND USES OF SAID DEVICE

(71) Applicant: ANVAJO GMBH, Dresden (DE)

(72) Inventor: Stefan FRAEDRICH, Dippoldiswalde (DE)

(73) Assignee: ANVAJO GMBH, Dresden (DE)

(21) Appl. No.: 16/628,051

(22) PCT Filed: May 28, 2018
(86) PCT No.: PCT/EP2018/063865

§ 371 Date: Jan. 2, 2020

(30) Foreign Application Priority Data
Jul. 5, 2017 (DE) 10 2017 211 478.9

(51) Int. Cl. B01L 3/00; C12Q 1/58; C12Q 1/26; G01N 21/75

(52) U.S. Cl. CPC B01L 3/502; C12Q 1/58; C12Q 1/26; G01N 33/49; B01L 2300/168; B01L 2400/0406; G01N 21/75

(57) ABSTRACT

The invention relates to a device and method for detecting a specific analyte in a liquid sample. The device that can be used in the method contains at least one fluid line, at least one receiving region for receiving a liquid sample, at least one enzymes region containing at least one determined enzyme and/or at least one acidification region containing at least one acid. The device also contains at least one reaction region used to form gas bubbles. The fluid line transports the liquid sample from the receiving region via the enzyme region and/or the acidification region to the reaction region by means of capillary forces and/or at least one micropump allowing fast, simple and cost-effective detection of a specific analyte in a liquid sample, the detection being carried out with a high level of sensitivity, specificity and precision. The invention further relates to uses of the device.

### BACKGROUND OF THE INVENTION

The invention relates to a device and a method for detecting a particular analyte in a liquid sample. The device which can be used in the method contains at least one fluid line, at least one receiving zone for the receiving of a liquid sample, at least one enzyme zone containing at least one particular enzyme and/or at least one acidification zone containing at least one acid. Furthermore, the device contains at least one reaction zone which is suitable for the formation of gas bubbles. The at least one fluid line is suitable for transporting the liquid sample by means of capillary forces and/or at least one micropump in the fluid line from the receiving zone to the reaction zone via the enzyme zone and/or the acidification zone. The device allows a rapid, simple and cost-effective detection of a particular analyte in a liquid sample, the detection being possible with high detection sensitivity, detection specificity and detection precision. Furthermore, uses of the device according to the invention are proposed.

Biomarkers are measurable parameters for biological processes that allow conclusions to be drawn regarding diseases and the physiological state. The best known biomarker molecules are especially hormones (e.g., T3/T4), metabolic products, blood sugar and also cholesterol and other blood fats.

Biomarkers are often present in very low concentrations and in a complex with a multiplicity of other proteins, and this not only makes characterization difficult, but also makes analysis costly and time-consuming. Commercially available, freely available rapid tests are usually based on a qualitative yes/no answer, but do not allow a quantitative assessment of the concentration of a particular analyte in a liquid sample.

In very many cases, serological methods are used for the detection of biomarkers as analytes. The disadvantages of said methods are high experimental complexity, since suitable antibodies must be chosen, the antibodies must be labeled (e.g., with a fluorescent dye) and the methods of determination are time-consuming. Furthermore, said methods of determination are negatively associated with high detection uncertainty.

Many of the methods known in the prior art for the qualitative or quantitative detection of a particular analyte in a liquid sample are based on the detection of high-specificity binding events between an epitope of the analyte to be determined (e.g., an epitope of an antigen) and a particular paratope on the detector molecule (e.g., a paratope of an antibody). Known methods for identifying biomarkers are, for example, enzyme-linked immunosorbent assay (ELISA), gel electrophoresis, surface plasmon resonance spectroscopy (SPR spectroscopy), the use of protein microarrays (e.g., mass-sensing BioCD protein array), surface-enhanced Raman spectroscopy, colorimetric or electrochemical methods, and fluorescence spectroscopy.

The current gold standard in biomarker diagnostics are immunoassays, in which there is a capture antibody immobilized on a solid phase and the reaction with the antigen is read with the aid of a secondary antibody. A disadvantage of said immunoassays is that nonspecific binding of other proteins to the sensor surface can affect the result in terms of providing a false positive and thus greatly distort the result.

Altogether, the current methods for identifying biomarkers are characterized by high expenditure in terms of time, high complexity in terms of apparatus, a need for technical personnel, low detection sensitivity, low detection specificity and low detection variance.

Proceeding from this, it is an object of the present invention to provide a device and a method which both allow a rapid, simple and cost-effective detection of a particular analyte in a liquid sample, and which both moreover allow high detection sensitivity, high detection specificity and high precision in detection. Furthermore, uses of such a device are to be proposed.

The object is achieved by the device, the method and the use.

### SUMMARY OF THE INVENTION

The invention provides a device for detecting a particular analyte in a liquid sample by means of enzyme-catalyzed and/or acid-mediated conversion of the analyte to yield at least one gas, containing
a) at least one fluid line;
b) at least one receiving zone for the receiving of a liquid sample;
c) i) at least one enzyme zone containing at least one particular enzyme suitable for catalyzing the conversion of the analyte to be determined to yield at least one gas; and/or
   ii) at least one acidification zone containing at least one acid; and
d) at least one reaction zone for the formation of gas bubbles, wherein the reaction zone contains or consists of a chamber which is fluidically connected to the at least one fluid line and which has liquid-tight walls;
   characterized in that the at least one fluid line is suitable for transporting the liquid sample from the receiving zone to the reaction zone via the at least one enzyme zone and/or the at least one acidification zone by means of capillary forces and/or at least one micropump in the fluid line.

The device according to the invention can thus comprise either at least one enzyme zone having the abovementioned features or at least one acidification zone having the abovementioned features or else comprise both at least one enzyme zone having the abovementioned features and at least one acidification zone having the abovementioned features.

Furthermore, the device can comprise a micropump suitable for transporting the liquid sample actively (i.e., by means of an input of energy) from the receiving zone to the reaction zone via the at least one enzyme zone and/or the at least one acidification zone. This active transport can support a passive transport (i.e., a transport by means of capillary forces). However, alternatively, the device (especially the at least one fluid line) can also be suitable, without a micropump, for transporting the liquid sample passively (i.e., solely by means of capillary forces) from the receiving zone to the reaction zone via the at least one enzyme zone and/or the at least one acidification zone.

In the case of transport by means of at least one micropump, the device according to the invention preferably comprises an energy source preferably selected from battery, accumulator, photovoltaic element and combinations thereof. However, the energy source necessary for the operation of the micropump can also be contained in an optical detection instrument and the device can draw the energy from the energy source when the device is arranged in or on the optical detection instrument.

The invention is based on a device for detecting biomarkers with the aid of gas-generating chemical reactions in a geometrically delimited sample chamber. By means of said device, particular analytes (e.g., biomarkers) can be characterized simply, rapidly and cost-effectively.

The device allows the determination of the analyte in the liquid sample with high detection sensitivity, since the analyte is converted catalytically by means of an enzyme or by means of acid to yield at least one gas and the production of just one mole of gas occupies a volume of 24.465·10⁻³ m³ (i.e., approx. 24.465 liters) at standard pressure (1013 hPa) and room temperature (25° C.). In other words, a gas volume of 24.465 nanoliters is released just with a conversion of one nanomole of analyte to yield one nanomole of gas, and this is readily measurable and quantifiable by means of established optical measurement methods. Consequently, the gas production causes a signal amplification which brings about high detection sensitivity.

Furthermore, the device according to the invention can provide high detection specificity, since the specificity of the enzyme-catalyzed conversion reaction depends on the specificity of the enzyme in relation to its substrate and/or since a particular sample to be analyzed can inevitably contain only one analyte which is converted by a reaction with an acid to yield a gas (e.g., in the case of blood as sample: conversion of HCO₃⁻ to yield CO₂+H₂O). In the case of an analyte binding event and analyte conversion by means of an enzyme, the specificity is even higher than the specificity of a multiplicity of analyte-antibody binding events, since, unlike in the case of analyte-antibody binding events, nonspecific analyte-enzyme binding events practically do not occur or hardly occur. Even if the analyte should bind nonspecifically to the enzyme, i.e., binds the enzyme not at the active site, there is no conversion of the analyte and there is no generation of a signal (in the form of a production of gas).

Furthermore, the device according to the invention makes it possible to provide low detection variance or high precision in detection (i.e., high proximity to the true value), since the at least one fluid line of the device according to the invention is suitable for transporting the liquid sample to the reaction zone by means of capillary forces and/or by means of a micropump. This means that the critical step of contacting the analyte with the enzyme and/or the acid, which step is responsible for the signal level generated, is “automated” and is effected in specified volumes. In other words, errors caused by manual mixing of particular volumes by a user (e.g., “pipetting errors”) are not applicable here. In other words, the accuracy of mixing of particular volumes is distinctly more constant from experiment to experiment in the case of the device according to the invention and the variance from experiment to experiment is distinctly smaller. Furthermore, the start of signal formation is exactly defined by the addition of the liquid sample to the receiving zone of the device (“automated” start) and there are thus no inaccuracies with regard to a reaction start brought about manually by a user. Consequently, the variance from measurement to measurement becomes smaller and the precision of detection is increased.

The device according to the invention can be characterized in that it contains multiple fluid lines, preferably multiple fluid lines suitable for transporting the liquid sample from the receiving zone to the reaction zone via at least one enzyme zone and/or at least one acidification zone by means of capillary forces and/or at least one micropump in the fluid line. Here, the fluid lines can each be connected to the at least one enzyme zone, acidification zone and/or reaction zone of the device according to the invention or, alternatively, each fluid line can be respectively connected to a separate enzyme zone, acidification zone and/or reaction zone. In the latter case, the simultaneous acquisition of multiple different analytes is possible with the device according to the invention. Here, the fluid lines can all each be connected to the at least one receiving zone of the device according to the invention or, alternatively, can each be connected to a separate receiving zone.

The device can contain multiple reaction zones, preferably multiple reaction zones for the formation of gas bubbles, wherein the reaction zones each contain or consist of a chamber, each fluidically connected to at least one fluid line and each having liquid-tight walls.

The chamber can contain at least one wall, preferably at least two opposing walls, which exhibits a transparency for light of a wavelength within a region selected from the group consisting of IR region, visible region, UV region and combinations thereof, preferably a transparency for light of a wavelength within the visible region.

Furthermore, the chamber can contain a wall, preferably at least two opposing walls, which exhibits a tightness for liquids, gases and combinations thereof, preferably a tightness for liquids.

The device can contain multiple chambers. In this case, the features of the chamber, as mentioned here, can apply to all the chambers of the device.

The device can be characterized in that the at least one receiving zone of the at least one fluid line is suitable for the receiving of a liquid sample selected from the group consisting of aqueous solutions containing or consisting of blood, urine, sputum, foodstuffs, river water, saltwater, seawater, groundwater, drinking water, wastewater and mixtures thereof.

The at least one enzyme zone can contain at least one enzyme selected from the group consisting of urease, lactate oxidase, lactate dehydrogenase, catalase, pyruvate decarboxylase, thyreoperoxidase and combinations thereof.

The at least one enzyme zone can contain at least one further enzyme, wherein the at least one further enzyme is preferably selected from the group consisting of catalase, pyruvate decarboxylase and combinations thereof.

The at least one enzyme zone can contain at least one cofactor of an enzyme, preferably NAD⁺.

The at least one enzyme zone can be arranged between the receiving zone for the receiving of a liquid sample and the reaction zone for the formation of gas bubbles or be arranged within the reaction zone for the formation of gas bubbles. If the device comprises multiple enzyme zones, such an arrangement can apply to all the enzyme zones of the device.

The at least one enzyme zone can contain the at least one enzyme and/or the at least one cofactor of the at least one enzyme in dry form, preferably in lyophilized form, or in aqueous form, preferably as an aqueous solution, aqueous suspension or aqueous gel.

The at least one enzyme zone can contain biological cells and/or cell lysate, wherein the biological cells and/or the cell lysate preferably contain/contains the at least one enzyme suitable for catalyzing the conversion of the analyte to be determined to yield at least one gas. The advantage of this embodiment is that an isolation (purification) of the enzyme is not necessary and that the enzyme is in an environment in which it can have a higher long-term stability than in purified form. Consequently, the device can have a higher long-term stability and can be provided more cost-effectively.

The at least one enzyme zone can contain at least urease. The at least one enzyme zone can contain lactate oxidase and catalase. The at least one enzyme zone can contain pyruvate decarboxylase. The at least one enzyme zone can contain lactate dehydrogenase and pyruvate decarboxylase.

The at least one fluid line can contain a membrane which preferably has a pore diameter of ≤20 μm, preferably ≤6 μm, particularly preferably ≤2 μm, in particular ≤100 nm, optionally ≤1 nm. The membrane can be suited to the removal of biological cells, preferably to the removal of blood cells. The membrane can be arranged between the receiving zone for the receiving of a liquid sample and the reaction zone for the formation of gas bubbles, preferably between the receiving zone for the receiving of a liquid sample and the enzyme zone and/or the acidification zone. If the device comprises multiple membranes, such an arrangement can apply to all the membranes of the device.

The at least one acidification zone preferably contains an acid selected from the group consisting of acids solid at room temperature and standard pressure, HCl, H₂SO₄, H₃PO₄ and mixtures thereof, preferably selected from the group consisting of acids solid at room temperature and standard pressure, particularly preferably citric acid, ascorbic acid, malic acid, stearic acid, palmitic acid, myristic acid, lauric acid and mixtures thereof.

The at least one acidification zone is preferably arranged between the receiving zone for the receiving of a liquid sample and the reaction zone for the formation of gas bubbles, particularly preferably within the reaction zone for the formation of gas bubbles. If the device comprises multiple acidification zones, such an arrangement can apply to all the acidification zones of the device.

The device, preferably the at least one fluid line, can contain at least one oxidation zone, wherein the at least one oxidation zone contains at least one oxidant, preferably at least one oxidant and at least one acid.

The at least one oxidation zone preferably contains an oxidant selected from the group consisting of potassium permanganate, manganese dioxide, NAD and mixtures thereof.

The at least one oxidation zone preferably contains an acid selected from the group consisting of acids solid at room temperature and standard pressure, HCl, H₂SO₄, H₃PO₄ and mixtures thereof, preferably selected from the group consisting of acids solid at room temperature and standard pressure, particularly preferably citric acid, ascorbic acid, malic acid, stearic acid, palmitic acid, myristic acid, lauric acid and mixtures thereof.

The at least one oxidation zone is preferably arranged between the receiving zone for the receiving of a liquid sample and the reaction zone for the formation of gas bubbles, particularly preferably between the enzyme zone and the reaction zone for the formation of gas bubbles. In particular, the at least one oxidation zone overlaps with the acidification zone at least regionally or is identical thereto.

The at least one fluid line can have a length of from 0.1 to 20 cm, preferably 0.5 to 10 cm, particularly preferably 1 to 5 cm. The at least one fluid line can have a width of from 0.05 to 20 mm, particularly preferably 0.1 to 10 mm, particularly preferably 1 to 5 mm, in particular 2 to 4 mm. The at least one fluid line can have a height of from 0.05 to 2 mm, particularly preferably 0.1 to 1 mm, particularly preferably 0.2 to 0.8 mm, in particular 0.4 to 0.6 mm. The at least one fluid line can have a maximum diameter within the range from 0.05 to 20 mm, preferably 0.1 to 10 mm, particularly preferably 1 to 5 mm, in particular 2 to 4 mm.

The particular analyte can be selected from the group consisting of small organic molecule having a mass of <500 Da, peptide, protein and mixtures thereof, preferably selected from the group consisting of hormone, metabolic product having a mass of <500 Da, carbohydrate, sterol, triglyceride, carboxylic acid, amide derivative of a carboxylic acid and mixtures thereof, particularly preferably selected from the group consisting of T3 hormone, T4 hormone, glucose, cholesterol, triglycerides from blood, lactic acid, urea and mixtures thereof.

The particular analyte can be an analyte which is a marker for a state selected from the group consisting of disease, water pollution, food contamination and combinations thereof.

The device can be arranged in or on an optical detection instrument, preferably in or on an optical microscope, wherein the optical detection instrument (e.g., the optical microscope) is particularly preferably configured to optically detect the reaction zone of the device and to carry out a qualitative and/or quantitative determination of the concentration of the analyte in the sample.

The optical detection instrument (e.g., the optical microscope) can be configured to qualitatively determine a presence of the analyte in the sample if the formation of gas bubbles occurs in the reaction zone.

The optical detection instrument (e.g., the optical microscope) can be configured to quantitatively determine the concentration of the analyte in the sample by means of a number and a volume of gas bubbles per unit of time, especially by means of the relationship that the product of number and volume of gas bubbles per unit of time is directly proportional to the concentration of the analyte in the sample.

The optical detection instrument can contain an energy source configured to supply the device according to the invention (e.g., at least one micropump thereof) with energy. Said energy source is preferably selected from energy from an electrical grid, battery, accumulator, photovoltaic element and combinations thereof.

The invention further provides a method for detecting a particular analyte in a liquid sample by means of enzyme-catalyzed and/or acid-mediated conversion of the analyte to yield at least one gas, comprising the steps of
a) applying a liquid sample possibly containing the analyte to be determined to a receiving zone of a device according to the invention;
b) optically detecting the reaction zone of the device as claimed in any of the preceding claims at a time point at which the enzyme-containing and/or acid-containing liquid sample has been transported from the fluid line to the reaction zone; and
c) assessing that the particular analyte is present in the sample if the formation of gas bubbles occurs in the reaction zone.

The method can be characterized in that the optical capture is effected by means of an optical detection instrument selected from the group consisting of camera, microscope, photometer, refractometer and combinations thereof, preferably by means of a microscope.

The method can encompass a quantitative determination of the concentration of the analyte in the sample, preferably by means of a determination of the number and the volume of gas bubbles per unit of time, particularly preferably by means of the relationship that the product of number and volume of gas bubbles per unit of time is directly proportional to the concentration of the analyte in the sample.

This can comprise the following steps:
a) applying at least one further liquid sample containing a known concentration of the analyte to be determined to a receiving zone of a device as claimed in any of the preceding claims;
b) optically detecting the reaction zone of the device as claimed in any of the preceding claims at a time point at which the enzyme-containing and/or acid-containing liquid sample has been transported from the fluid line to the reaction zone; and
c) quantitatively determining the concentration of the analyte in the sample, preferably by means of a determination of the number and the volume of gas bubbles per unit of time, particularly preferably by means of the relationship that the product of number and volume of gas bubbles per unit of time is directly proportional to the concentration of the analyte in the sample.

Furthermore, there is proposed the use of the device according to the invention for the in vitro diagnosis of a disease, wherein the liquid sample is preferably selected from the group consisting of blood, urine, sputum and mixtures thereof. There is further proposed the use of the device according to the invention for the quality control of foodstuffs, preferably for the quality control of a food liquid, particularly preferably for the quality control of wine, fruit juices and combinations thereof. There is moreover proposed the use of the device according to the invention for the testing of water quality, preferably river water quality, saltwater quality, seawater quality, drinking water quality and combinations thereof.

### DESCRIPTION OF THE DRAWINGS

It is intended that the subject matter of the invention be more particularly elucidated on the basis of the following figures and examples, without wishing to restrict said subject matter to the specific embodiments depicted here.

FIG. 1 shows the enzymes and/or acid for conversion of particular biomarkers which can be contained by the device according to the invention;

FIG. 2 shows the device;

FIG. 3 show the top plan view of the device; and

FIG. 4 shows a cross-section view of a detection instrument suitable for performing an optical capture of the reaction zone of the device.

### DETAILED DESCRIPTION OF THE DRAWINGS

FIG. 1 shows, by way of example, possible enzymes and/or acids (optionally with oxidant) for the conversion of particular analytes (biomarkers) which can be contained by the device according to the invention and which can be used in the method according to the invention. In particular, the figure also describes the respective chemical reactions forming the basis of gas evolution.

FIG. 2 shows, by way of example, a possible device 1 according to the invention for the detection of a particular analyte in a liquid sample 2 by means of enzyme-catalyzed conversion of the analyte to yield at least one gas 7. The device 1 contains at least one fluid line 3 which is fluidically connected to at least one receiving zone 4 for the receiving of a liquid sample 2 (e.g., blood) and fluidically connected to at least one enzyme zone 5, the enzyme zone 5 containing at least one particular enzyme which catalyzes the conversion of the analyte to be determined to yield at least one gas 7. Furthermore, the device 1 contains at least one reaction zone 6 for the formation of gas bubbles 7, the reaction zone 6 having a fluidic connection to the fluid line 3 and being otherwise delimited by a gas-tight wall 10. The device 1 is characterized in that the at least one fluid line 3 is suitable for transporting the liquid sample 2 from the receiving zone 4 to the reaction zone 6 by means of capillary forces, the at least one particular enzyme being, in this case, cotransported from the enzyme zone 5 at least in part into the reaction zone 6. In this embodiment, the device 1 further contains a membrane 8 suited to the removal of biological cells (e.g., a membrane for the separation of blood cells from blood plasma) between the receiving zone 4 and the enzyme zone 5. Said membrane 8 ensures that no biological cells get from the fluid line 3 as far as the enzyme zone 5 by means of capillary forces. Moreover, said device 1 additionally contains an acidification zone 9 which contains at least one acid (e.g., HCl) and, in this case, coincides with the reaction zone 6. The acidification zone 9 ensures that the liquid sample, which contains enzyme in the reaction zone 6, is acidified. In the case of gases which dissolve in the liquid sample under acid formation (e.g., formation of H₂CO₃ in the case of the gas CO₂), the acidification shifts the equilibrium in the direction of gas formation and a stronger (quantitative) escape of CO₂ is thus effected. In brief, the presence of the acidification zone 9 can distinctly increase the detection sensitivity in the case of this type of gas.

FIG. 3 shows a top view of a reaction chamber 6 of a device according to the invention. What is depicted is a temporal course during the reaction, with the gas bubbles 7 growing in the course of time to form larger gas bubbles 7′. Besides the final volume expansion of the gas bubbles 7 to form the larger gas bubbles 7′, it is also possible to utilize the volume increase over time from the state of the original gas bubbles 7 up to the state of the larger gas bubbles 7′.

FIG. 4 shows a cross section of a detection instrument suitable for performing an optical capture of the reaction zone of the device according to the invention and for carrying out and outputting a quantitative determination of the concentration of the analyte in the sample. The gas bubbles 7 which are formed in the reaction zone 6 of the device according to the invention are completely illuminated by electromagnetic radiation 12 of a substantially coherent or noncoherent light source 11 (e.g., an LED, an OLED, a laser and/or a gas discharge lamp). An image sensor 13 (e.g., in the form of a diode array) which is situated under the reaction zone 6 captures the optical image 14 generated by the gas bubbles 7 (i.e., the characteristic interference pattern thereof). From said optical image 14, it is possible by means of a data processing program to calculate the product of number and volume of gas bubbles per unit of time, which is directly proportional to the reaction rate and to the concentration of the analyte in the sample. An optical device (not depicted here) can likewise additionally be situated between the image sensor and the reaction zone.

### EXAMPLE 1—FUNDAMENTALS IN RELATION TO THE METHOD FOR DETECTING A PARTICULAR ANALYTE IN A LIQUID SAMPLE

The maximum reaction rate vₘₐₓ of an enzymatic reaction depends on the temperature and the pH of the solution in which the reaction is carried out. For a particular, constant pH, vₘₐₓ is dependent on the ambient temperature of the device according to the invention. If the ambient temperature is also constant (e.g., constant 25° C. room temperature), vₘₐₓ assumes a completely definite value. In this case, the measured reaction rate v of substrate (analyte) is dependent on the concentration of substrate (analyte) in the solution (Michaelis-Menten theory). What is applicable here is the equation

v=(vₘₐₓ·[Analyte])/(kₘ+[Analyte])

where [Analyte] is the analyte concentration and kₘ represents the Michaelis-Menten constant of the enzyme.

In the case of a known vₘₐₓ under particular conditions and known Michaelis-Menten constant kₘ, it is thus possible by means of the measurement of the reaction rate of the substrate (analyte) to deduce the concentration of substrate (analyte) in the liquid sample.

The reaction rate is directly proportional to the gas volume produced per unit of time, i.e., proportional to the product of number and volume of detected gas bubbles. What is thus applicable is the relationship

v˜[Number(gas bubbles)·Volume(gas bubbles)]

Many of the gas bubbles which arise dissolve poorly in the liquid sample, meaning that the aforementioned relationship is fully applicable.

Since carbon dioxide is very highly soluble in aqueous solution and dissociates in part to yield carbonic acid, pH change is used for the conversion into the gaseous aggregate state. Since the sample chamber is delimited in all three dimensions by a gas-impermeable layer, gas bubbles are formed which cannot escape from the sample chamber. The amount and size of the gas bubbles is analyzed with the aid of an optical method. The amount and size of the gas bubbles correlates with quantity of the analyte in the sample.

### EXAMPLE 2—METHOD FOR DETECTING UREA IN A LIQUID SAMPLE

For the detection of the analyte urea, the device according to the invention contains the enzyme urease in the enzyme zone. As can be seen in FIG. 1, the enzyme urease catalyzes the conversion of urea to yield the two gases ammonia and carbon dioxide.

An acidification zone on the device according to the invention ensures that, firstly, the carbon dioxide which arises does not go into solution as H₃O⁺ and HCO₃⁻, but escapes quantitatively. As a result, the sensitivity of detection of the device for urea is increased.

Moreover, the acidification causes the ammonia which arises to preferentially go into solution as NH₄⁺ and OH⁻. However, since ammonia has anyway a high tendency to dissolve in aqueous media even at neutral pH, the acidification barely shifts the equilibrium in the direction of dissolved ammonia. In other words, the acidification barely reduces the production of ammonia gas, meaning that, with regard to the production of ammonia gas, the acidification barely causes an adverse effect on detection sensitivity.

The product of number and volume (amount) of carbon dioxide gas bubbles is thus dependent on the urea concentration in the liquid sample used. The amount of gas bubbles produced decreases with falling urea concentration.

The amount of gas bubbles can be recorded by means of a software-controlled microscope and be evaluated (e.g., with regard to their number and their geometric properties).

By way of example, the use of the device in so-called “point of care” urea diagnostics shall be described:

A drop of blood is applied to the at least one receiving zone of the device. The drop can, for example, be received directly from a (freshly) punctured finger of a person. In this case, the device advantageously contains a plasma-separating membrane, with the result that the blood cells are held back and only blood plasma can advance as far as the enzyme zone. The enzyme zone advantageously contains the enzyme urease in lyophilized form, since the long-term stability of the enzyme is very high in this form and, as a result, the device also ensures a long usability.

The blood plasma is drawn, along the fluid line by means of capillary forces, from the receiving zone to the enzyme zone, where it meets the lyophilized urease, which goes into solution in the blood plasma. The mixture of blood plasma and urease is promptly transported, via the acidification zone by means of capillary force, to the reaction zone, where carbon dioxide is released by the decomposition of urea. The amount of gas bubbles formed is recorded with the aid of an optical method (e.g., a software-controlled microscope), and this allows conclusions to be drawn regarding the concentration of urea in the blood drop used.

### EXAMPLE 3—METHOD FOR DETECTING LACTATE IN A LIQUID SAMPLE

The enzyme lactate oxidase selectively converts lactate into pyruvate and hydrogen peroxide (see FIG. 1). In a second step, the hydrogen peroxide reacts, owing to its strongly reducing effect, with a potassium permanganate solution slightly acidified with sulfuric acid. The redox reaction leads, firstly, to the decolorization of the potassium permanganate solution, which has an intense purple color, and, secondly, to the formation of oxygen (see FIG. 1).

For reaction of the hydrogen peroxide with the potassium permanganate solution acidified with sulfuric acid, the device thus further requires an oxidation zone containing at least one oxidant (e.g., MnO₄⁻ due to dissolved KMnO₄) and at least one acid (H₃O⁺, for example due to dissolved H₂SO₄).

The product of number and volume (amount) of oxygen gas bubbles is thus dependent on the lactate concentration in the liquid sample used. The amount of gas bubbles produced decreases with falling lactate concentration.

The amount of gas bubbles can be recorded by means of a software-controlled microscope and be evaluated (e.g., with regard to their number and their geometric properties).

1. A device for detecting a particular analyte in a liquid sample by means of at least one conversion of the analyte selected from the group consisting of an enzyme-catalyzed conversion of the analyte and an acid-mediated conversion of the analyte to yield, at least one gas, the device comprising
at least one fluid line;
at least one receiving zone for the receiving of a liquid sample;
at least one zone selected from the group consisting of at least one enzyme zone comprising at least one particular enzyme suitable for catalyzing the conversion of the analyte to be determined to yield at least one gas, and
at least one acidification zone comprising at least one acid; and
at least one reaction zone for the formation of gas bubbles, wherein the reaction zone comprises or consists of a chamber which is fluidically connected to the at least one fluid line and which has liquid-tight walls;
wherein the at least one fluid line is suitable for transporting the liquid sample from the receiving zone to the reaction zone via at least one zone selected from the group consisting of the at least one enzyme zone and/or the at least one acidification zone, by means of at least one selected from the group consisting of capillary forces and at least one micropump in the fluid line.

2. The device as claimed in claim 1, wherein the device comprises at least one selected from the group consisting of
multiple fluid lines; and
multiple reaction zones.

3. The device as claimed in claim 1, wherein the chambers comprises at least one wall, which exhibits at least one selected from the group consisting of
a transparency for light of a wavelength within a region selected from the group consisting of IR region, visible region, UV region and combinations thereof; and
a tightness for liquids, gases and combinations thereof.

4. The device as claimed in claim 1, wherein the at least one receiving zone is suitable for receiving a liquid sample selected from the group consisting of aqueous solutions comprising or consisting of blood, urine, sputum, foodstuffs, river water, saltwater, seawater, groundwater, drinking water, wastewater and mixtures thereof.

5. The device as claimed in claim 1, wherein the at least one enzyme zone comprises at least one selected from the group consisting of
at least one enzyme selected from the group consisting of urease, lactate oxidase, lactate dehydrogenase, catalase, pyruvate decarboxylase, thyreoperoxidase and combinations thereof;
at least one further enzyme; and
at least one cofactor of an enzyme.

6. The device as claimed in claim 1, wherein in that the at least one enzyme zone comprises the at least one enzyme and/or at least one cofactor of the at least one enzyme, or both,
in dry form; or
in aqueous form.

7. The device as claimed in claim 1, wherein the at least one enzyme zone comprises at least one selected from the group consisting of biological cells and cell lysate.

8. The device as claimed in claim 1, wherein in that the at least one fluid line comprises a membrane which has at least one selected from the group consisting of a pore diameter of ≤20 μm;
a suitability for the removal of biological cells, and
an arrangement between the receiving zone for the receiving of a liquid sample and the reaction zone for the formation of gas bubbles.

9. The device as claimed in claim 1, wherein the acidification zone has at least one selected from the group consisting of
an acid selected from the group consisting of acids solid at room temperature and standard pressure, HCl, H₂SO₄, H₃PO₄ and mixtures thereof; and
an arrangement between the receiving zone for the receiving of a liquid sample and the reaction zone for the formation of gas bubbles or is arranged within the reaction zone for the formation of gas bubbles.

10. The device as claimed in claim 1, wherein the at least one fluid comprises at least one oxidation zone, wherein the oxidation zone comprises at least one oxidant.

11. The device as claimed in claim 1, wherein the at least one fluid line has at least one selected from the group consisting of
a length of from 0.1 to 20 cm;
a width of from 0.05 to 20 mm;
a height of from 0.05 to 2 mm; and
a maximum diameter within the range from 0.05 to 20 mm.

12. The device as claimed in claim 1, wherein the particular analyte is at least one is selected from the group consisting of a small organic molecule having a mass of <500 Da, peptide, protein and mixtures thereof; and
an analyte which is a marker for a state selected from the group consisting of disease, water pollution, food contamination and combinations thereof.

13. The device as claimed in claim 1, wherein the device is arranged in or on an optical detection instrument.

14. A method for detecting a particular analyte in a liquid sample by means of at least one conversion of the analyte selected from the group consisting of an enzyme-catalyzed conversion of the analyte and an acid-mediated conversion of the analyte, to yield at least one gas, the method comprising the steps of
applying a liquid sample possibly comprising the analyte to be determined to a receiving zone of a device, wherein the device is a device for detecting a particular analyte in a liquid sample by means of at least one conversion of the analyte selected from the group consisting of an enzyme-catalyzed conversion of the analyte and an acid-mediated conversion of the analyte, to yield at least one gas, wherein the device comprises
at least one fluid line;
at least one receiving zone for the receiving of a liquid sample;
at least one zone selected from the group consisting of at least one enzyme zone comprising at least one particular enzyme suitable for catalyzing the conversion of the analyte to be determined to yield at least one gas, and at least one acidification zone comprising at least one acid; and
at least one reaction zone for the formation of gas bubbles, wherein the reaction zone comprises or consists of a chamber which is fluidically connected to the at least one fluid line and which has liquid-tight walls;
wherein the at least one fluid line is suitable for transporting the liquid sample from the receiving zone to the reaction zone via at least one zone selected from the group consisting of the at least one enzyme zone and the at lease one acidification zone, by means of at least one selected from the group consisting of capillary force and at least one micropump in the fluid line;
optically detecting the reaction zone of the device at a time point at which the liquid sample comprising at least one of the group consisting of an enzyme and acid has been transported from the fluid line to the reaction zone; and
assessing that the particular analyte is present in the sample if the formation of gas bubbles occurs in the reaction zone.

15. The method as claimed in claim 14, wherein the optical capture is effected by means of an optical detection instrument selected from the group consisting of camera, microscope, photometer, refractometer and combinations thereof.

16. The method as claimed in claim 14, wherein the method encompasses a quantitative determination of the concentration of the analyte in the sample.

17. The method as claimed in claim 14, in further comprising the steps of applying at least one further liquid sample comprising a known concentration of the analyte to be determined to a receiving zone of the device;
optically detecting the reaction zone of the device at a time point at which the liquid sample comprising at least one selected from the group consisting of the enzyme and acid has been transported from the fluid line to the reaction zone; and
quantitatively determining the concentration of the analyte in the sample.

18. (canceled)

19. A method in which a device for detecting a particular analyte in a liquid sample by means of at least one conversion of the analyte selected from the group consisting of enzyme-catalyzed conversion of the analyte and acid-mediated conversion of the analyte, to yield at least one gas, is used for at least one of in vitro diagnosis of a disease, quality control of foodstuffs and testing of water quality,
wherein the used device comprises
at least one fluid line;
at least one receiving zone for the receiving of a liquid sample;
at least one zone selected from the group consisting a at least one enzyme zone comprising at least one particular enzyme suitable for catalyzing the conversion of the analyte to be determine to yield at least one gas, and at least one acidification zone comprising at least one acid; and
at least one reaction zone for the formation of gas bubbles, wherein the reaction zone comprises or consists of a chamber which is fluidically connected to the at least one fluid line and which has liquid-tight walls;
wherein the at least one fluid line is suitable for transporting the liquid sample from the receiving zone to the reaction via at least one zone selected from the group consisting of the at least one enzyme zone and the at least one acidification zone, by means of at least one selected from the group consisting of capillary forces and at least one micropump in the fluid line.
